(19) United States
(12) Patent Application Publication
(10) Pub. No.: US 2017/0215693 A1
(43) Pub. Date: Aug. 3, 2017

(54) ENDOSCOPE SYSTEM

(71) Applicant: OLYMPUS CORPORATION, Tokyo (JP)

(72) Inventors: Yasuhito KURA, Tokyo (JP); Kazuki HONDA, Tokyo (JP); Takeo SUZUKI, Tokyo (JP); Takanori WATANABE, Tokyo (JP)

(73) Assignee: OLYMPUS CORPORATION, Tokyo (JP)

(21) Appl. No.: 15/485,352

(22) Filed: Apr. 12, 2017

Related U.S. Application Data
(63) Continuation of application No. PCT/JP2015/079181, filed on Oct. 15, 2015.

(30) Foreign Application Priority Data
Nov. 18, 2014 (JP) 2014-233871

(51) Int. Cl. A61B 1/00; G06T 7/11; G02B 23/24; H04N 5/225; G02B 23/12; A61B 1/04; G06T 11/60

(52) U.S. Cl. CPC A61B 1/0005; A61B 1/00177; A61B 1/04; G06T 7/11; G06T 11/60; H04N 5/2256; G02B 23/12; G02B 23/2484; H04N 2005/2255

(57) ABSTRACT

An endoscope system includes: an insertion portion inserted into a subject; a first image acquisition section provided on the insertion portion and configured to acquire a first image from a first region of an object; a second image acquisition section provided on the insertion portion and configured to acquire a second image from a second region of the object including a region adjacent to the first region; and an image processing section configured to execute image processing by setting a first display region and a second display region adjacent to the first display region in a display section configured to display an image, disposing the first image in a first display region, disposing part of the second image in the first display region adjacent to the first image, and disposing remaining part of the second image in a second display region.

### CROSS REFERENCE TO RELATED APPLICATION

This application is a continuation application of PCT/JP2015/079181 filed on Oct. 15, 2015 and claims benefit of Japanese Application No. 2014-233871 filed in Japan on Nov. 18, 2014, the entire contents of which are incorporated herein by this reference.

### BACKGROUND OF THE INVENTION

1. Field of the Invention

The present invention relates to an endoscope system, and particularly, to an endoscope system configured to acquire object images of a forward field of view and a lateral field of view.

2. Description of the Related Art

Conventionally, an endoscope is widely used in a medical field and an industrial field. The endoscope includes illumination means and observation means on a distal end side of an insertion portion. The endoscope is inserted into a subject, and the endoscope can observe and inspect inside of the subject.

In recent years, a multi-camera endoscope that can observe two or more directions is proposed as disclosed for example in Japanese Patent Application Laid-Open Publication No. 2013-544617, the multi-camera endoscope including a lateral field of view in which a lateral side of the insertion portion is an observation field of view, in addition to a forward field of view in which a forward side of the insertion portion is an observation field of view. An inspector can use the multi-camera endoscope to observe two forward and lateral directions at the same time to observe the inside of the subject with a wide-angle field of view.

### SUMMARY OF THE INVENTION

An aspect of the present invention provides an endoscope system including: a first image acquisition section configured to acquire a first image from a first region of an object; a second image acquisition section configured to acquire a second image from a second region of the object including a region adjacent to the first region; an image synthesis section configured to divide the second image into an adjacent image adjacent to the first image and an image of another region, generate a synthetic image by synthesizing the first image and the adjacent image so as to dispose the adjacent image adjacent to the first image, and generate the image of the other region as an image different from the synthetic image; and an image output section configured to output the images from the image synthesis section, wherein the image output section is provided with: a multiple-display mode of individually converting the synthetic image and the image of the other region into display signals, outputting, to a first display apparatus, the display signal obtained by converting the synthetic image, and outputting, to a second display apparatus, the display signal obtained by converting the image of the other region; and a single-display mode of generating, based on the synthetic image and the image of the other region, a display signal indicating a video including the synthetic image and the image of the other region disposed separately from each other and outputting the display signal to a display apparatus, and the image output section switches one of the multiple-display mode and the single-display mode to output the display signal.

### BRIEF DESCRIPTION OF THE DRAWINGS

FIG. 1 is a configuration diagram showing a configuration of an endoscope system according to a first embodiment;

FIG. 2 is a schematic diagram showing a configuration of a processor configured to cause three display apparatuses to display observation images according to the first embodiment;

FIG. 3 is a diagram describing image regions cut out from a captured image region according to the first embodiment;

FIG. 4 is a diagram for describing a state in which an image region for forward field of view is converted according to the first embodiment;

FIG. 5 is a diagram for describing a state in which an image region for first lateral field of view is converted according to the first embodiment;

FIG. 6 is a diagram for describing a state in which an image region for second lateral field of view is converted according to the first embodiment;

FIG. 7 is a diagram describing a procedure of image processing according to the first embodiment;

FIG. 8 is a diagram showing a state in which the observation images are displayed on three display apparatuses according to the first embodiment;

FIG. 9 is a diagram showing a state in which the observation images are displayed on one display apparatus according to the first embodiment;

FIG. 10 is a schematic diagram showing a configuration of the processor configured to cause display apparatuses to display the observation images according to a first modification of the first embodiment;

FIG. 11 is a diagram describing a procedure of image processing according to the first modification of the first embodiment;

FIG. 12 is a diagram showing a state in which the observation images are displayed on three display apparatuses according to the first modification of the first embodiment;

FIG. 13 is a diagram showing a state in which the observation images are displayed on one display apparatus according to the first modification of the first embodiment;

FIG. 14 is a schematic diagram showing a configuration of the processor configured to cause the display apparatuses to display the observation images according to a second modification of the first embodiment;

FIG. 15 is a diagram describing a procedure of image processing according to the second modification of the first embodiment;

FIG. 16 is a diagram showing a state in which the observation images are displayed on three display apparatuses according to the second modification of the first embodiment;

FIG. 17 is a diagram showing a state in which the observation images are displayed on one display apparatus according to the second modification of the first embodiment;

FIG. 18 is a schematic diagram showing a configuration of the processor configured to cause the display apparatuses to display the observation images according to a third modification of the first embodiment;

FIG. 19 is a diagram describing a procedure of image processing according to the third modification of the first embodiment;

FIG. 20 is a diagram showing a state in which the observation images are displayed on three display apparatuses according to the third modification of the first embodiment;

FIG. 21 is a diagram showing a state in which the observation images are displayed on one display apparatus according to the third modification of the first embodiment;

FIG. 22 is a schematic diagram showing a configuration of the processor configured to cause the display apparatuses to display the observation images according to a fourth modification of the first embodiment;

FIG. 23 is a diagram describing a procedure of image processing according to the fourth modification of the first embodiment;

FIG. 24 is a diagram showing a state in which the observation images are displayed on three display apparatuses according to the fourth modification of the first embodiment;

FIG. 25 is a diagram showing a state in which the observation images are displayed on one display apparatus according to the fourth modification of the first embodiment;

FIG. 26 is a schematic diagram showing a configuration of the processor configured to cause the display apparatuses to display the observation images according to a fifth modification of the first embodiment;

FIG. 27 is a diagram describing a procedure of image processing according to the fifth modification of the first embodiment;

FIG. 28 is a diagram showing a state in which the observation images are displayed on three display apparatuses according to the fifth modification of the first embodiment;

FIG. 29 is a diagram showing a state in which the observation images are displayed on one display apparatus according to the fifth modification of the first embodiment;

FIG. 30 is a diagram showing five display apparatuses displaying subject images taken by an endoscope according to a second embodiment;

FIG. 31 is a perspective view showing a configuration of a distal end portion of the endoscope according to a modification of the second embodiment;

FIG. 32 is a diagram showing five display apparatuses displaying the subject images taken by the endoscope according to the modification of the second embodiment;

FIG. 33 is a diagram showing one display apparatus displaying the subject images taken by the endoscope according to the modification of the second embodiment;

FIG. 34 is a diagram showing an image recording section and the display apparatuses displaying the endoscopic images according to a third embodiment; and

FIG. 35 is a perspective view of a distal end portion 11 of an insertion portion 10 provided with a unit for lateral observation according to a fourth embodiment.

### DETAILED DESCRIPTION OF THE PREFERRED EMBODIMENTS

Hereinafter, an endoscope apparatus that is the present invention will be described. Note that in the following description, drawings based on respective embodiments are schematic drawings, and a relationship between thickness and width of each part, a ratio of the thickness of respective parts, and the like are different from the reality. The relationship or the ratio of dimensions between the drawings may also be different in part of the drawings.

Note that an endoscope in the following description of configuration is inserted into an upper or lower digestive organ of a living body, and the endoscope is a so-called flexible endoscope including a flexible insertion portion in an example described below. However, the endoscope is not limited to this, and the endoscope is a technique that can also be applied to a so-called rigid endoscope including a rigid insertion portion used for surgery.

### First Embodiment

First, an endoscope system of an aspect of the present invention will be described based on the drawings.

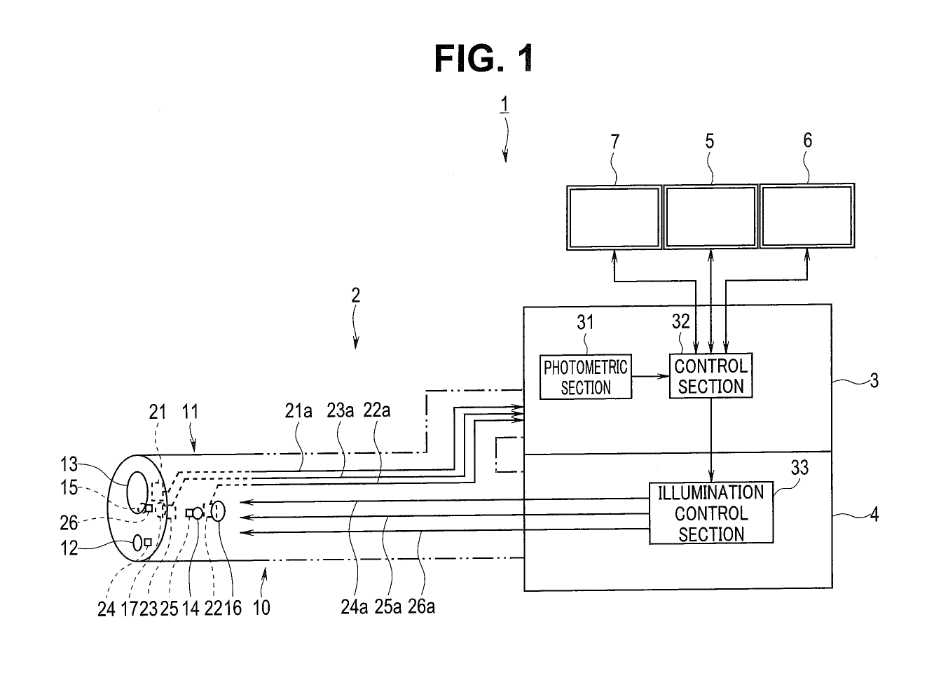

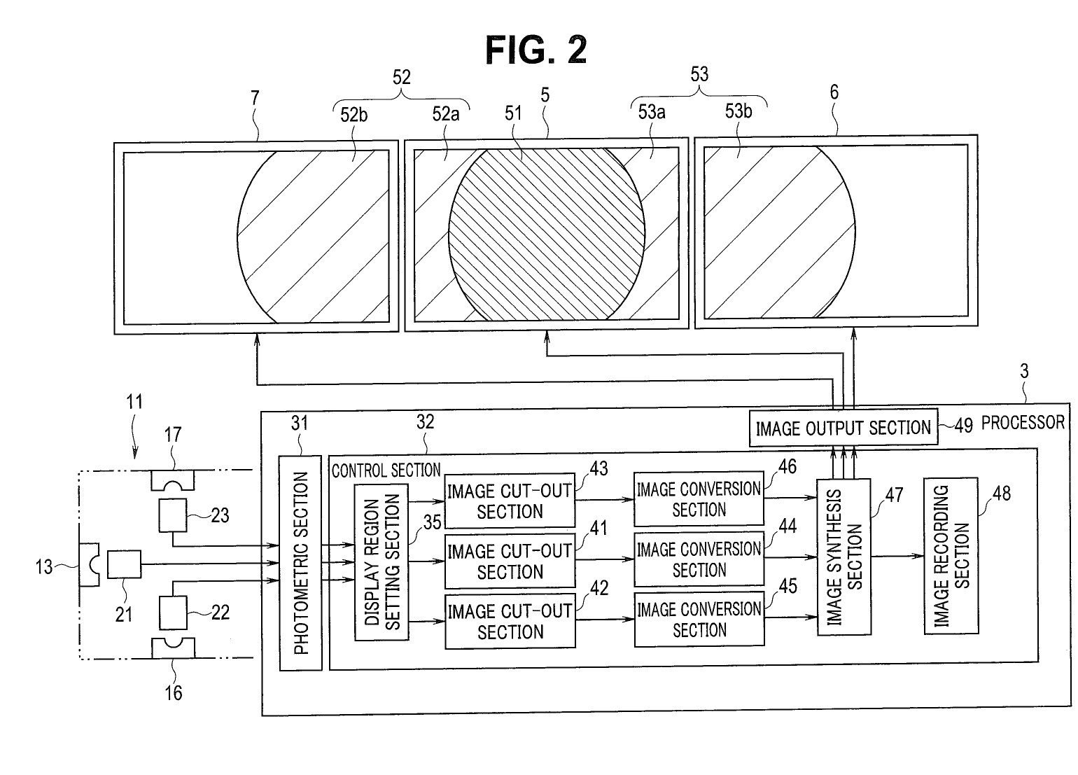

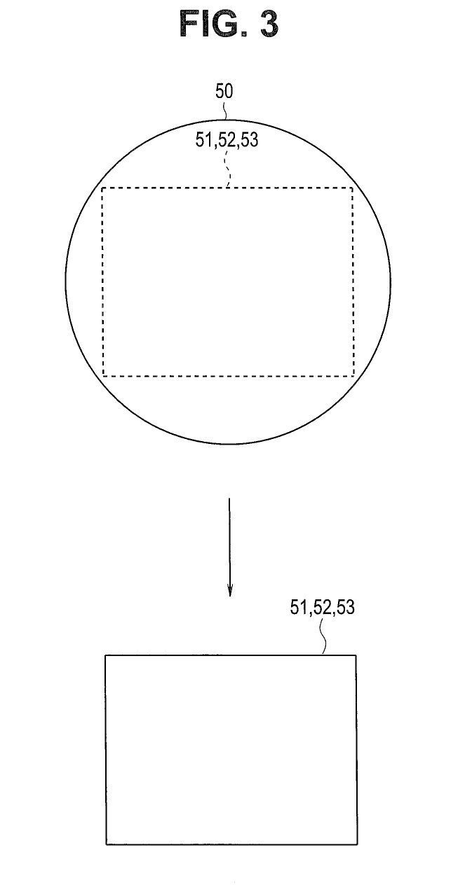

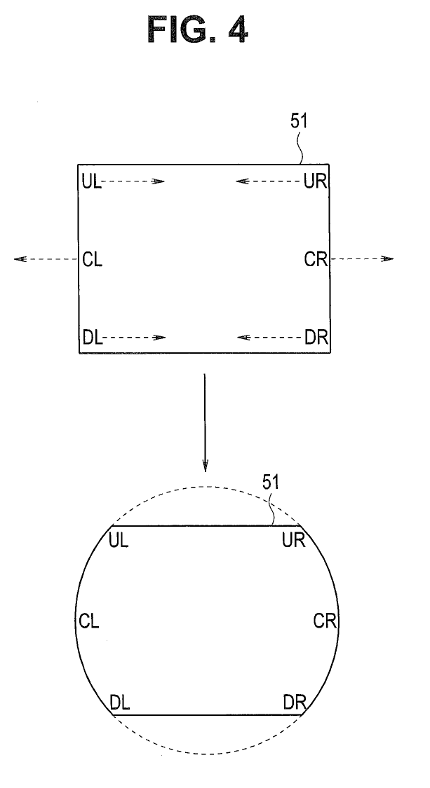

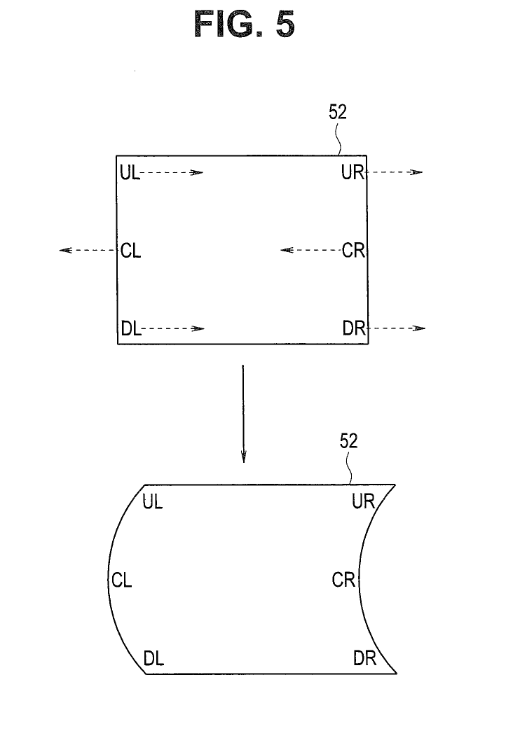

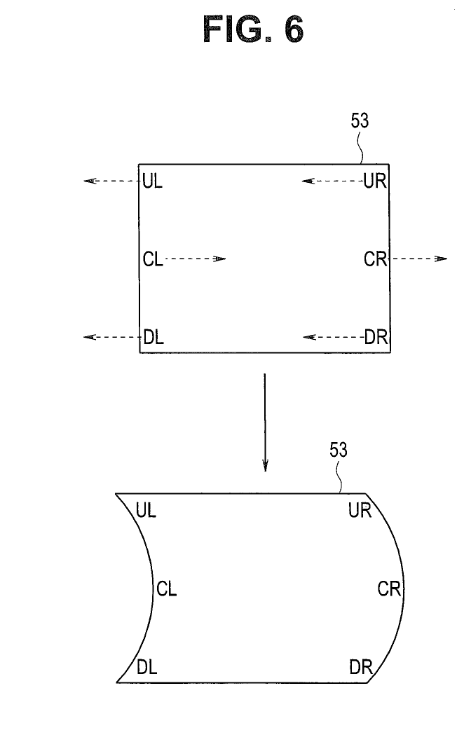

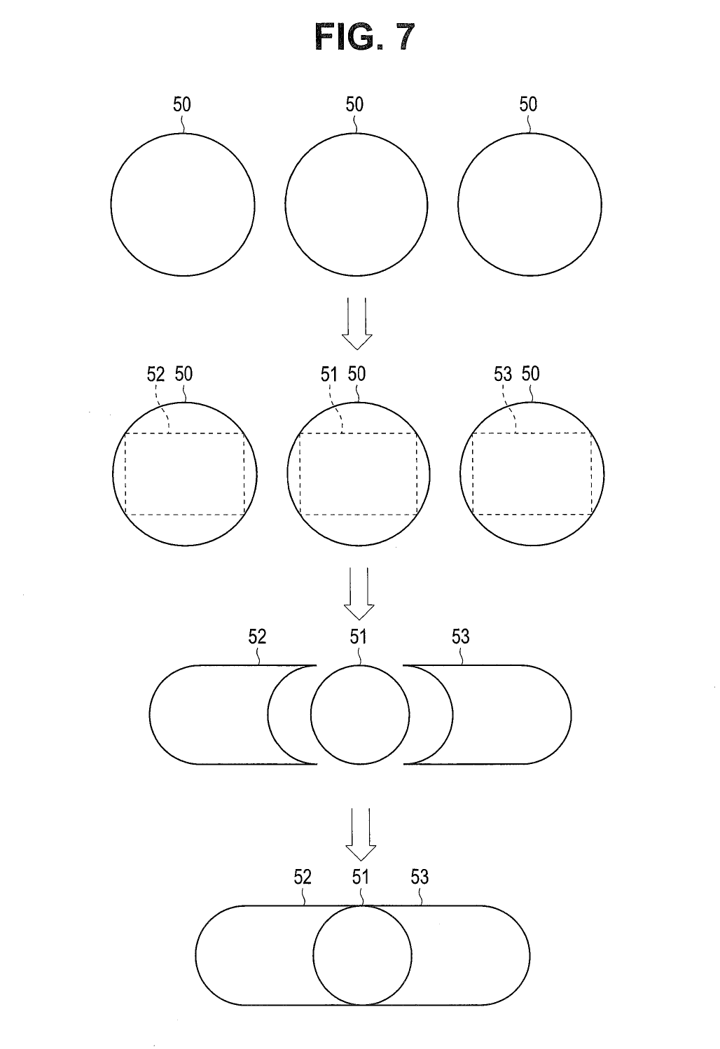

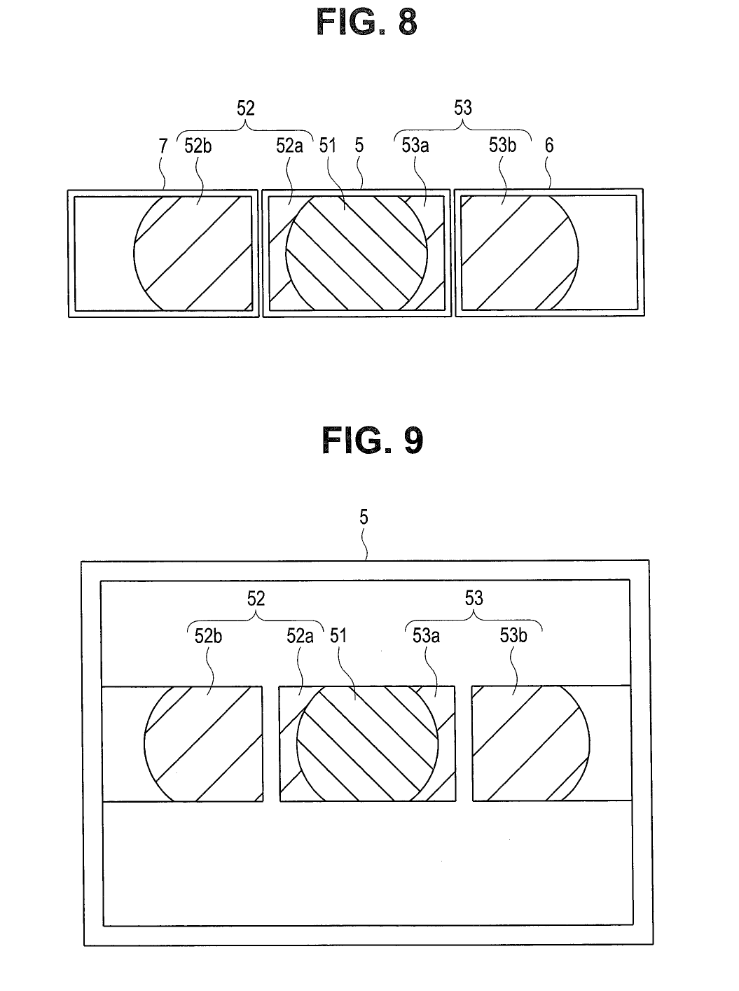

FIG. 1 is a configuration diagram showing a configuration of an endoscope system according to the present embodiment. FIG. 2 is a schematic diagram showing a configuration of a processor configured to cause three display apparatuses to display observation images. FIG. 3 is a diagram describing image regions cut out from a captured image region. FIG. 4 is a diagram for describing a state in which an image region for forward field of view is converted. FIG. 5 is a diagram for describing a state in which an image region for first lateral field of view is converted. FIG. 6 is a diagram for describing a state in which an image region for second lateral field of view is converted. FIG. 7 is a diagram describing a procedure of image processing. FIG. 8 is a diagram showing a state in which the observation images are displayed on three display apparatuses. FIG. 9 is a diagram showing a state in which the observation images are displayed on one display apparatus.

An endoscope system 1 shown in FIG. 1 includes an endoscope 2, a processor 3, a light source apparatus 4, and monitors 5, 6, and 7 as three display apparatuses here.

The endoscope 2 includes a flexible insertion portion 10 inserted into a subject and an operation portion not shown and is connected to the processor 3 and the light source apparatus 4 through endoscope cables not shown.

In the endoscope 2, a distal end portion 11 of the insertion portion 10 is provided with an illumination window (first illumination portion) 12 and an observation window (first image acquisition section) 13 for forward field of view, two illumination windows (second illumination portions) 14 and 15 for lateral field of view, and two observation windows (second image acquisition sections) 16 and 17.

That is, the endoscope 2 includes two illumination windows 14 and 15 here in addition to the illumination window 12 and includes two observation windows 16 and 17 here in addition to the observation window 13.

The illumination window 14 and the observation window 16 are for a first lateral field of view, and the illumination window 15 and the observation window 17 are for a second lateral field of view. The plurality of, two here, observation windows 16 and 17 are disposed in a circumferential direction of the insertion portion 10 at substantially uniform angles.

Note that the distal end portion 11 of the insertion portion 10 includes a distal end rigid member not shown. The illumination window 12 is provided on a distal end surface of the distal end rigid member, and the two illumination windows 14 and 15 are provided on a side surface of the distal end rigid member.

In the distal end portion 11, an image pickup unit 21 for forward field of view is located on a back side of the observation window 13 for forward field of view. In the distal end portion 11, an image pickup unit 22 for first lateral field of view is located on a back side of the observation window 16, and an image pickup unit 23 for second lateral field of view is located on a back side of the observation window 17.

Each of the three image pickup units 21, 22, and 23 that are image pickup sections includes an image pickup device such as a CCD and a CMOS, is electrically connected to the processor 3, is controlled by the processor 3, and outputs an image pickup signal to the processor 3. Therefore, each of the image pickup units 21, 22, and 23 is an image pickup section configured to photoelectrically convert an image (object image).

The observation window 13 is disposed in a direction of insertion of the insertion portion 10 in the distal end portion 11 of the insertion portion 10, and the observation windows 16 and 17 are disposed in an outer diameter direction of the insertion portion 10 in a side surface portion of the insertion portion 10.

That is, the observation window 13 is a first image acquisition section provided on the distal end portion 11 of the insertion portion 10 and configured to acquire a first object image that is a first image from a forward region that is a first region.

Each of the observation windows 16 and 17 is a second image acquisition section provided on the distal end portion 11 of the insertion portion 10 and configured to acquire a second object image that is a second image from a lateral region that is a second region as a region in a left and right direction here that is different from the forward region.

In other words, the first object image that is the first image is an object image of the first region including the forward direction of the insertion portion substantially parallel to a longitudinal direction of the insertion portion 10, and the second object image that is the second image is an object image of the section region in the left and right direction including the lateral direction of the insertion portion substantially orthogonal to the longitudinal direction of the insertion portion 10. Note that the second regions may be regions in an up and down direction provided at positions where the positions of the observation windows 16 and 17 are rotated substantially 90 degrees about a center axis of the insertion portion 10.

An illumination light emitting device 24 for forward field of view is located on a back side of the illumination window 12 for forward field of view. An illumination light emitting device 25 for first lateral field of view is located in the distal end portion 11, on a back side of the illumination window 14. An illumination light emitting device 26 for second lateral field of view is located in the distal end portion 11, on a back side of the illumination window 15.

The illumination light emitting devices (hereinafter, called illumination light emitting devices) 24, 25, and 26 are, for example, light emitting diodes (LEDs).

Therefore, the illumination window 12 corresponding to the illumination light emitting device 24 is an illumination portion configured to emit illumination light in the forward direction, and the illumination windows 14 and 15 corresponding to the illumination light emitting devices 25 and 26, respectively, are illumination portions configured to emit the illumination light in the lateral direction.

A photometric section 31 and a control section 32 are built in the processor 3. Three image pickup signals outputted from the three image pickup units 21, 22, and 23 are inputted to the control section 32 through image pickup cables 21a, 22a, and 23a. The control section 32 generates and synthesizes three endoscopic images based on the three image pickup signals and outputs the three endoscopic images to the three monitors 5, 6, and 7.

An illumination control section 33 is built in the light source apparatus 4. The illumination control section 33 is controlled by the control section 32 of the processor 3.

The illumination control section 33 is a circuit configured to control amounts of emitted light and ON/OFF of the illumination light emitting devices 24, 25, and 26. The illumination control section 33 outputs control signals to the respective illumination light emitting devices 24, 25, and 26 through signal lines 24a, 25a, and 26a. The illumination control section 33 also controls the amount of light of each of the illumination light emitting devices 24, 25, and 26 based on a light adjustment signal from the control section 32.

The illumination control section 33 also controls light emission timing of ON and OFF of each illumination light emitting device. That is, the illumination control section 33 is an illumination control section configured to control emission of illumination light in the forward direction and emission of illumination light in the lateral direction at different predetermined timings.

The endoscope system 1 configured as described above synthesizes the object images acquired by the respective image pickup units 21, 22, and 23 provided on the distal end portion 11 of the endoscope 2 and displays the object images on the three monitors 5, 6, and 7.

In this case, the synthetic images generated by the control section 32 of the processor 3 that is an image generation section are displayed on the three monitors 5, 6, and 7.

More specifically, as shown in FIG. 2, the control section 32 of the processor 3 is provided with a display region setting section 35, three cut-out sections 41, 42, and 43, three image conversion sections 44, 45, and 46, an image synthesis section 47, and an image recording section 48 such as various memories. Note that the image recording section 48 may be able to be attached to and detached from the processor 3.

The image pickup signals photoelectrically converted by the three image pickup units 21, 22, and 23 built in the distal end portion 11 of the endoscope 2 are inputted to the three cut-out sections 41, 42, and 43, respectively, through the photometric section 31 and the display region setting section 35.

More specifically, the image pickup signal from the image pickup unit 21 for forward field of view is inputted to the cut-out section 41 for forward field of view. The image pickup signal from the image pickup unit 22 for first lateral field of view is inputted to the cut-out section 42 for first lateral field of view. The image pickup signal from the image pickup unit 23 for second lateral field of view is inputted to the cut-out section 43 for second lateral field of view.

That is, the three cut-out sections 41, 42 and 43 are provided in the control section 32 for the respective field of view directions of the endoscope 2.

As shown in FIG. 3, the three cut-out sections 41, 42, and 43 convert the respectively inputted image pickup signals to image pickup signals by cutting out (masking) only image information of predetermined image regions 51, 52, and 53 that are rectangular here, respectively, from a substantially circular captured image region 50.

The three cut-out sections 41, 42, and 43 then output, to the electrically connected image conversion sections 44, 45, and 46, the image pickup signals obtained by cutting out only the image information of the set predetermined image regions 51, 52, and 53.

The three image conversion sections 44, 45, and 46 convert images based on the inputted image pickup signals into set shapes.

More specifically, as shown in FIG. 4, the image conversion section 44 for forward field of view converts the cut-out predetermined image region 51 into a circular shape and then masks upper and lower parts here to distort both side portions into a convex, curved, arc shape.

In this case, the image conversion section 44 converts the image data into an image shape, in which upper left and right corner portions UL and UR and lower left and right corner portions DL and DR of the image region 51 are distorted inward (toward center), and left and right intermediate portions CL and CR in the up and down direction of the image region 51 are distorted outward (opposite side of center).

As shown in FIG. 5, the image conversion section 45 for first lateral field of view distorts a left side of the cut-out predetermined image region 52 into a convex curved shape and distorts a right side into a concave curved shape.

In this case, the image conversion section 45 converts the image data into an image shape, in which upper left and right corner portions UL and UR and lower left and right corner portions DL and DR of the image region 52 are distorted to the right, and left and right intermediate portions CL and CR in the up and down direction of the image region 51 are distorted to the left.

More specifically, as shown in FIG. 4, the image conversion section 44 for forward field of view deforms the cut-out predetermined image region 51 into a round image and then makes an adjustment, such as masking the upper and lower parts, to distort both side portions into a convex, curved, arc shape, such as an elliptical shape, a circular shape, and a barrel shape.

In this case, the image conversion section 46 converts the image data into an image shape, in which upper left and right corner portions UL and UR and lower left and right corner portions DL and DR of the image region 53 are deformed to the left, and left and right intermediate portions CL and CR in the up and down direction of the image region 51 are deformed to the right.

Note that the image conversion sections 44, 45, and 46 distort the image regions 51, 52, and 53, respectively, such that the converted convex curved shapes of the left and right side portions of the image region 51 in the forward field of view direction correspond to the converted concave curved shapes of one of the left and right side portions of the two image regions 52 and 53 in each lateral field of view direction.

The respective image conversion sections 44, 45, and 46 output the converted image data of the image regions 51, 52, and 53 to the image synthesis section 47.

The image synthesis section 47 then synthesizes the inputted three image data of the image regions 51, 52, and 53 into one image in which the concave curved shapes of the image regions 52 and 53 correspond to the convex curved shapes of the left and right side portions of the image region 51.

In this case, the image synthesis section 47 synthesizes the data by setting the image region 51 that is the forward field of view at the center, setting the image region 52 for first lateral field of view on the left of the image region 51 and adjacent to the image region 51, and setting the image region 52 for second lateral field of view on the right of the image region 51 and adjacent to the image region 51.

The synthesized image data is converted by the image synthesis section 47 with a magnification designated in advance and converted to display signals by an image output section 49. The display signals are outputted to the three monitors 5, 6, and 7.

In this case, the image synthesis section 47 simultaneously outputs, to the image recording section 48, synthetic image data obtained by synthesizing the three image data of the image regions 51, 52, and 53. Note that the image recording section 48 records the inputted synthetic image data.

As shown in FIG. 2, in a display region of the monitor 5 installed at the center, an observation image of the image region 51 in the forward field of view direction is displayed at the center, and partial observation images 52a and 53a adjacent to the image region 51 in the two image regions 52 and 53 in the respective lateral field of view directions are displayed.

That is, the monitor 5 includes a first display region for displaying the observation image of the image region 51 and a second display region for displaying the observation images 52a and 53a.

The display region setting section 35 of the processor 3 recognizes each of the first display region and the second display region and allocates the observation images by setting the display such that the observation image of the image region 51 is disposed in the first display region and the observation images 52a and 53a are disposed in the second display region.

A user can arbitrarily set a display ratio of the observation image of the image region 51 in the forward field of view direction in this case and the partial observation images 52a and 53a of the two image regions 52 and 53 in the respective lateral field of view directions displayed in the second display region adjacent to the image region 51 displayed in the first display region.

A remaining partial observation image 52b excluding the observation image 52a in the image region 52 in the first lateral field of view direction is displayed on the monitor 6 installed on the left of the monitor 5.

Note that a remaining partial observation image 53b excluding the observation image 53a in the image region 53 in the second lateral field of view direction is displayed on the monitor 7 installed on the right of the monitor 5.

That is, in the endoscope system 1 of the present embodiment, the control section 32 of the processor 3 causes the three image cut-out sections 41, 42, and 43 to cut out round images of the three captured image regions 50 in a rectangular shape as shown in FIG. 7.

In the control section 32, the image conversion section 44 converts the forward image region 51 into a round shape, such as an elliptical shape, a circular shape, and a barrel shape, the image conversion sections 45 and 46 convert the two lateral image regions 52 and 53 into concave shapes, and the image synthesis section 47 synthesizes the three image regions 51, 52, and 53.

In this way, the endoscope system 1 displays the forward image region 51 in the first display region of the monitor 5 and displays the partial observation images 52a and 53a of the lateral image regions 52 and 53 in the second display region as shown in FIG. 8.

Note that the remaining observation images 52b and 53b of the lateral image regions 52 and 53 are displayed on the monitors 6 and 7.

In this way, dimensions in a horizontal direction of the lateral images displayed on the respective display regions are substantially correctly displayed on the three monitors 5, 6, and 7.

The endoscope system 1 further records the processed images in the image recording section 48 provided on the control section 32 of the processor 3.

As described, part of the observation image in each lateral field of view direction is also displayed on the monitor 5 disposed at the center in addition to the observation image in the forward field of view direction, and the subject images are displayed without being wasted in the endoscope system 1 of the present embodiment.

Therefore, the endoscope system 1 can efficiently display the observation image of the forward field of view and the observation images of the respective lateral fields of view, and continuity can be felt in the information of the subject images. That is, the images give the impression that the observation image in the forward field of view direction and the observation images in the respective lateral field of view directions extend to the left and right from the center of the monitor 5 at the center.

Even if the user concentrates on the monitor 5 disposed at the center, the observation images of the respective lateral fields of view are also displayed, and the user also receives the information in the lateral direction of the subject images. Therefore, neglect of a lesion is prevented more than in the past. Particularly, the user obtains the information of the subject images in the lateral direction. Therefore, the necessity to carefully view all of the monitors 5, 6, and 7 is reduced, and a degree of fatigue is reduced.

As described, the endoscope system 1 can reduce the fatigue of the user by reducing the waste of the observation images displayed on the monitors 5, 6, and 7 that are screens and improving the visibility of the observation images.

The fact that the second region (lateral field of view) is different from the first region (forward field of view) indicates that optical axes are in different directions. To prevent significantly losing the field of view when the images are cut out, the ranges of the forward field of view and the lateral fields of view may partially overlap at boundary parts of the forward field of view and the lateral fields of view. The ranges may be adjacent to each other with almost no gap, or the ranges may be separated at a slight interval without an overlapped range.

Image processing of reducing unnatural feeling may be further executed by performing a boundary treatment or the like for smoothly connecting the part where the first object image and the second object are adjacent to each other.

Note that although the regions are set to display one display part on the screen of each of the three monitors 5, 6, and 7, the processor 3 may set a plurality of, for example three, display parts on one screen of one monitor as shown in FIG. 9, and a switching function for operation in another operation mode may be included, in which the forward field of view image and the lateral field of view images are displayed on the plurality of display parts, respectively.

### First Modification

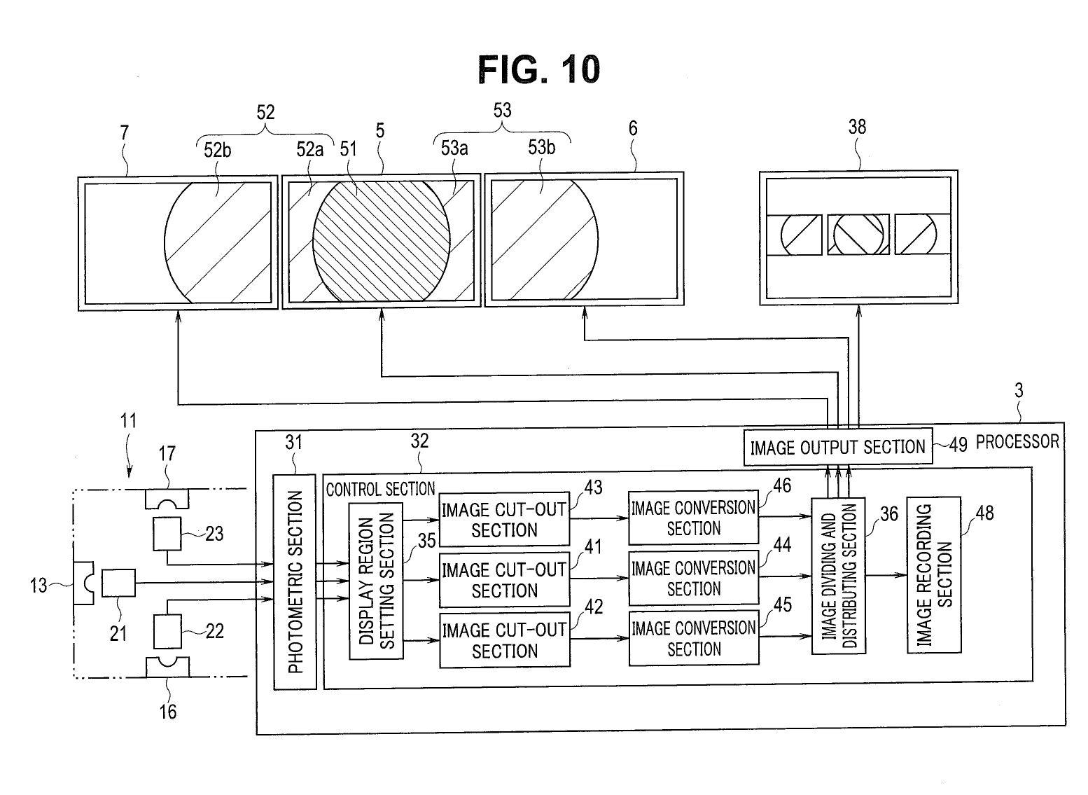

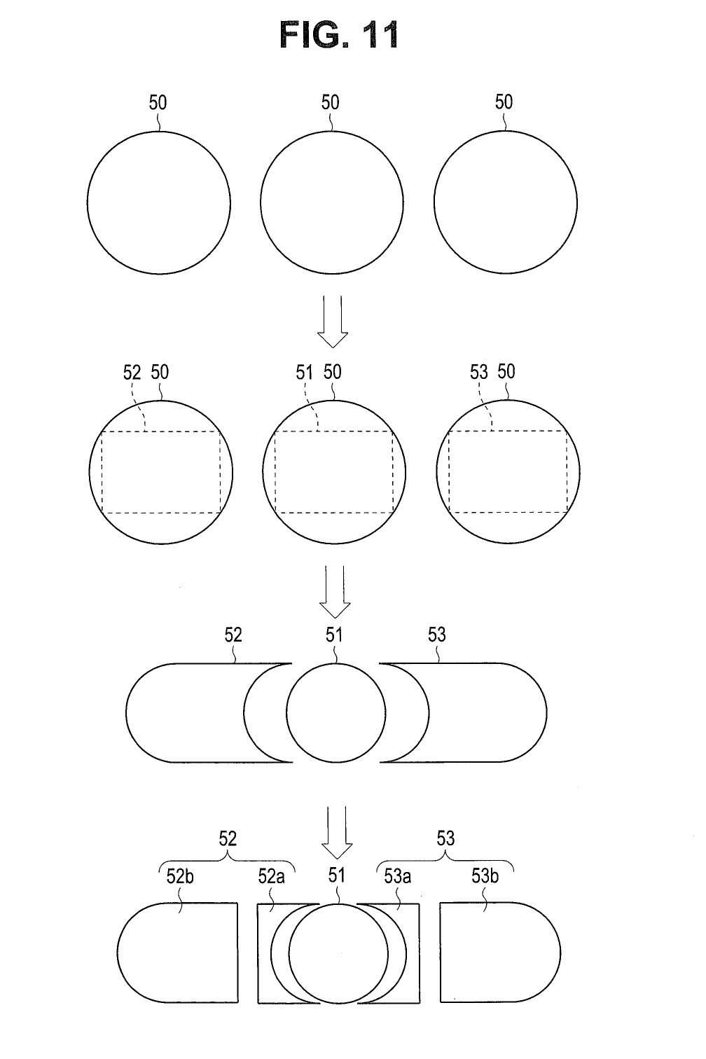

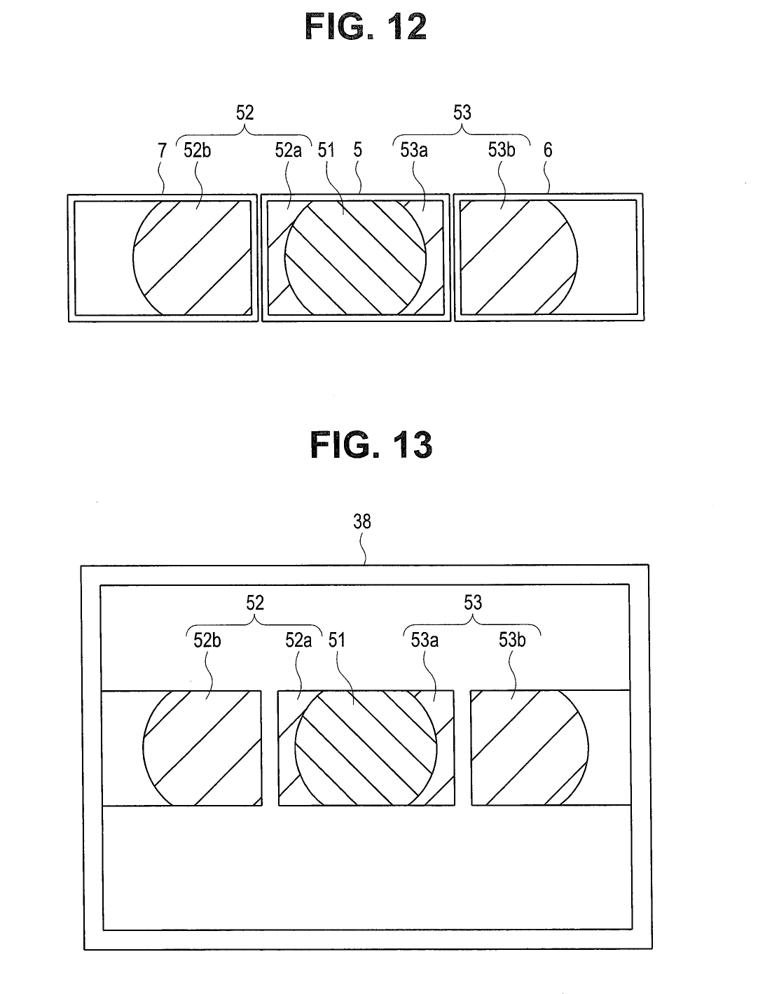

FIG. 10 is a schematic diagram showing a configuration of the processor configured to cause display apparatuses to display the observation images according to a first modification. FIG. 11 is a diagram describing a procedure of image processing of the first modification. FIG. 12 is a diagram showing a state in which the observation images are displayed on three display apparatuses according to the first modification. FIG. 13 is a diagram showing a state in which the observation images are displayed on one display apparatus according to the first modification.

In the endoscope system 1 of the present modification, the control section 32 of the processor 3 is provided with an image dividing and distributing section 36 in place of the image synthesis section 47, and a monitor 38 is further provided in addition to the three monitors 5, 6, and 7 as shown in FIG. 10.

In the endoscope system 1 configured in this way, the three image cut-out sections 41, 42, and 43 of the control section 32 of the processor 3 cut out round images of the three captured image regions 50 in a rectangular shape as shown in FIG. 11.

In the control section 32, the image conversion section 44 converts the forward image region 51 into a round shape, such as an elliptical shape, a circular shape, and a barrel shape, and the image conversion sections 45 and 46 convert the two lateral image regions 52 and 53 into concave shapes.

Subsequently, in the control section 32, the image dividing and distributing section 36 divides parts of the lateral image regions 52 and 53 to convert the image regions 52 and 53 to allow displaying the image regions 52 and 53 along with the forward image region 51 (note that synthesis of only the parts is also possible).

In this way, as shown in FIG. 12, the endoscope system 1 displays the forward image region 51 in the first display region of the monitor 5 among the three monitors 5, 6, and 7 and displays the partial observation images 52a and 53a of the lateral image regions 52 and 53 in the second display region. The endoscope system 1 displays the remaining lateral observation images 52b and 53b on the monitors 6 and 7.

The endoscope system 1 also records the processed images in the image recording section 48 provided on the control section 32 of the processor 3.

According to the configuration, the endoscope system 1 can prevent part of both lateral images from being hidden at the boundaries of the monitor 5 and the monitors 6 and 7.

Note that although the regions are also set to display one display part on the screen of each of the three monitors 5, 6, and 7 in the endoscope system 1 of the present modification, the processor 3 may set a plurality of, for example three, display parts on one screen of one monitor 38 as shown in FIG. 13, and a switching function for operation in another operation mode may be included, in which the forward field of view image and the lateral field of view images are displayed on the plurality of display parts, respectively.

### Second Modification

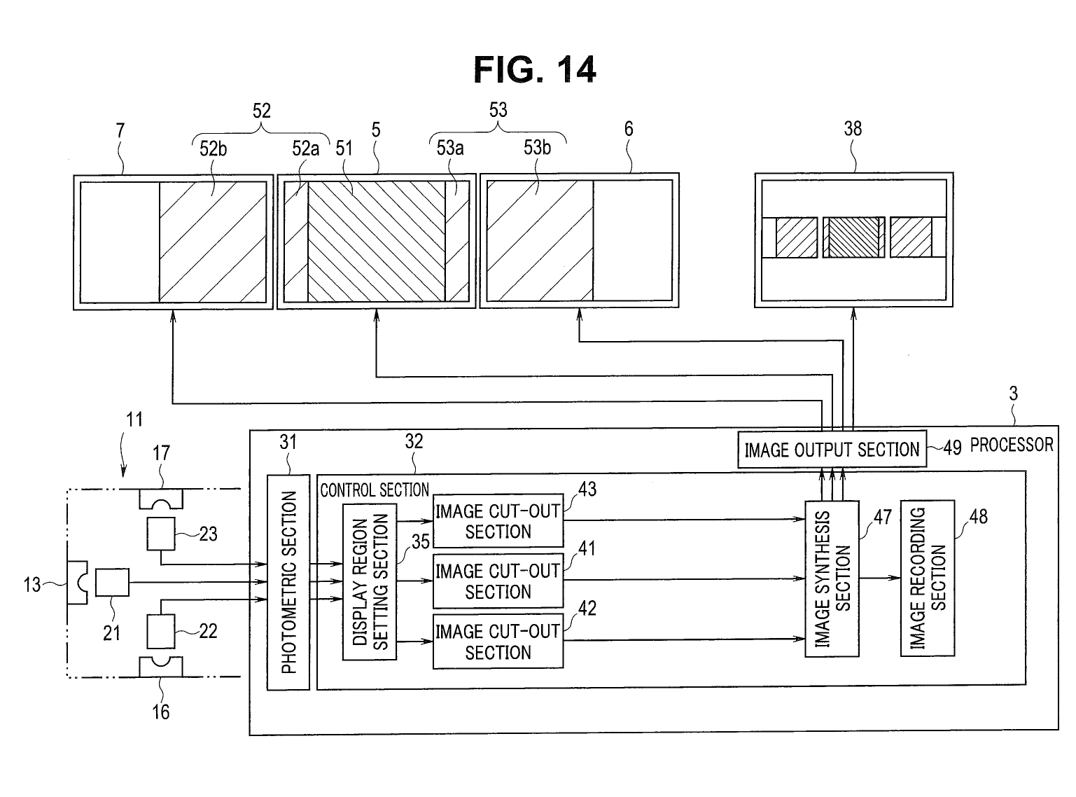

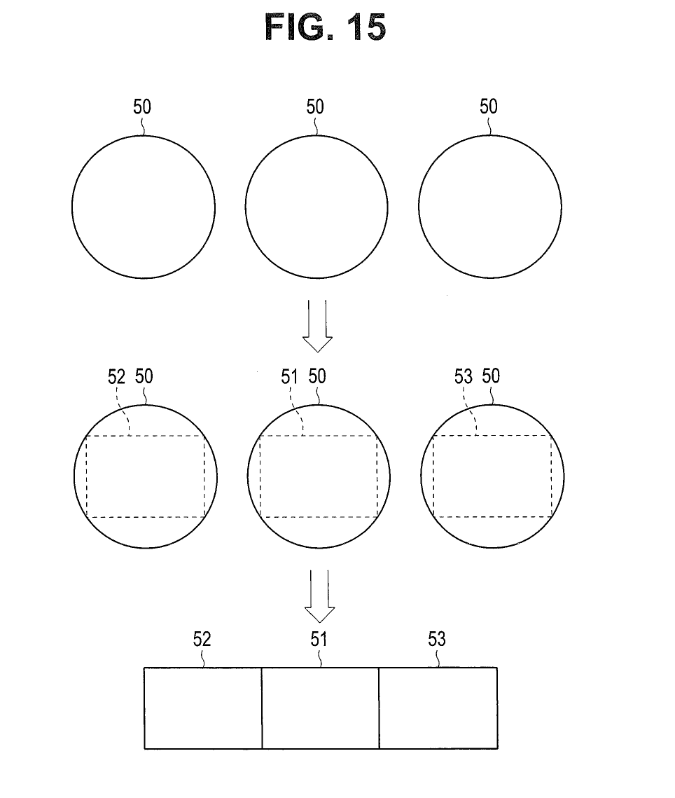

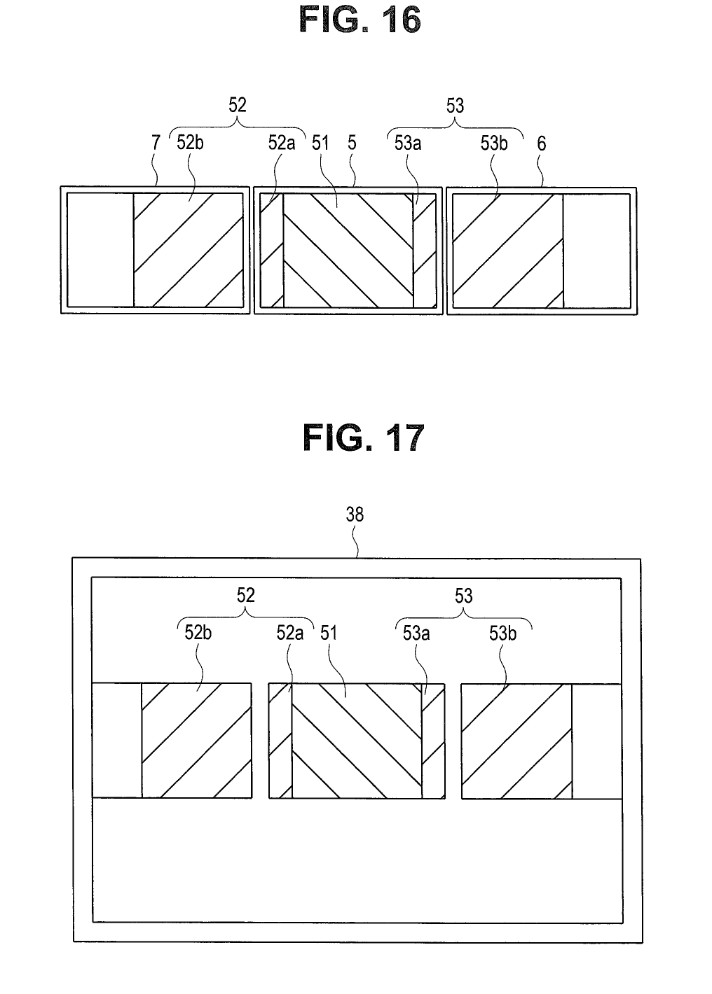

FIG. 14 is a schematic diagram showing a configuration of the processor configured to cause the display apparatuses to display the observation images according to a second modification. FIG. 15 is a diagram describing a procedure of image processing of the second modification. FIG. 16 is a diagram showing a state in which the observation images are displayed on three display apparatuses according to the second modification. FIG. 17 is a diagram showing a state in which the observation images are displayed on one display apparatus according to the second modification.

In the endoscope system 1 of the present modification, the image conversion sections 44, 45, and 46 are not provided on the control section 32 of the processor 3, and the monitor 38 is further provided in addition to the three monitors 5, 6, and 7 as shown in FIG. 14.

In the endoscope system 1 of the present modification configured in this way, the three image cut-out sections 41, 42, and 43 of the control section 32 of the processor 3 cut out the round images of the three captured image regions 50 in a rectangular shape as shown in FIG. 15.

In the control section 32, the image synthesis section 47 synthesizes the three cut-out rectangular forward and lateral image regions 51, 52, and 53. As shown in FIG. 16, the forward image region 51 is displayed in the first display region of the monitor 5 among the three monitors 5, 6, and 7, and the partial observation images 52a and 53a of the lateral image regions 52 and 53 are displayed in the second display region. The remaining lateral observation images 52b and 53b are displayed on the monitors 6 and 7.

The endoscope system 1 also records the processed images here in the image recording section 48 provided on the control section 32 of the processor 3.

According to the configuration, the endoscope system 1 can substantially correctly display the dimensions in the horizontal direction of the lateral images displayed in the respective display regions.

Note that although the regions are also set to display one display part on the screen of each of the three monitors 5, 6, and 7 in the endoscope system 1 of the present modification, the processor 3 may set a plurality of, for example three, display parts on one screen of one monitor 38 as shown in FIG. 17, and a switching function for operation in another operation mode may be included, in which the forward field of view image and the lateral field of view images are displayed on the plurality of display parts, respectively.

### Third Modification

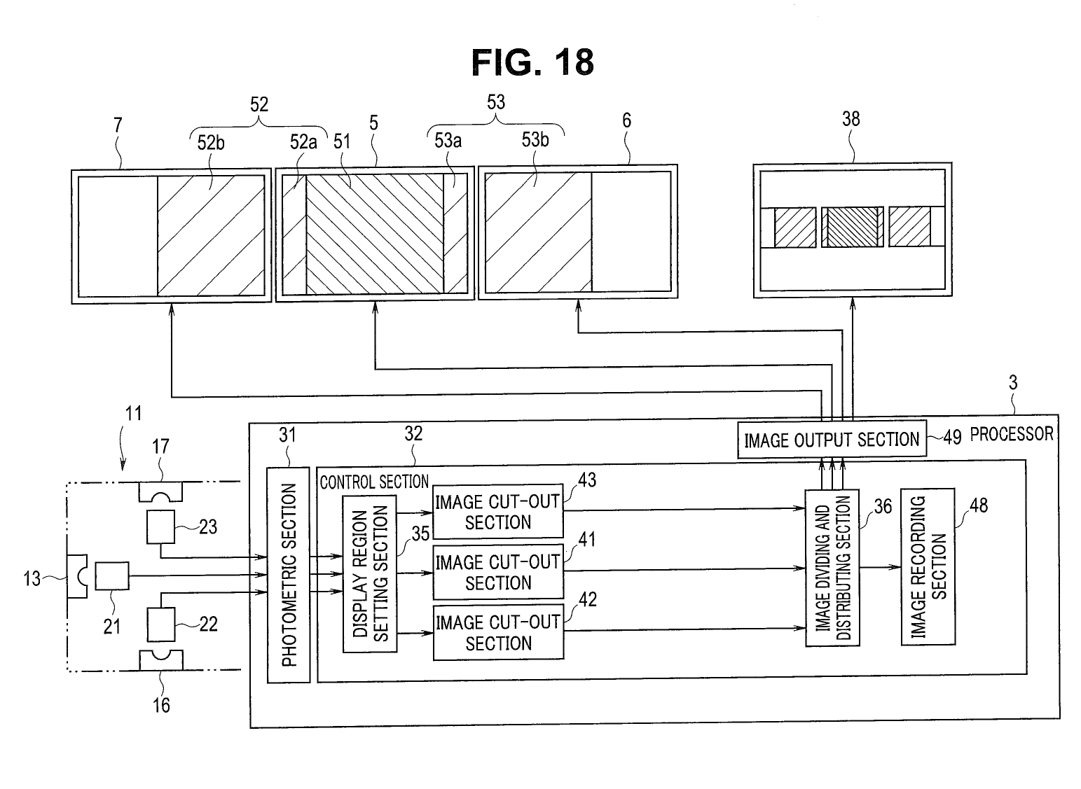

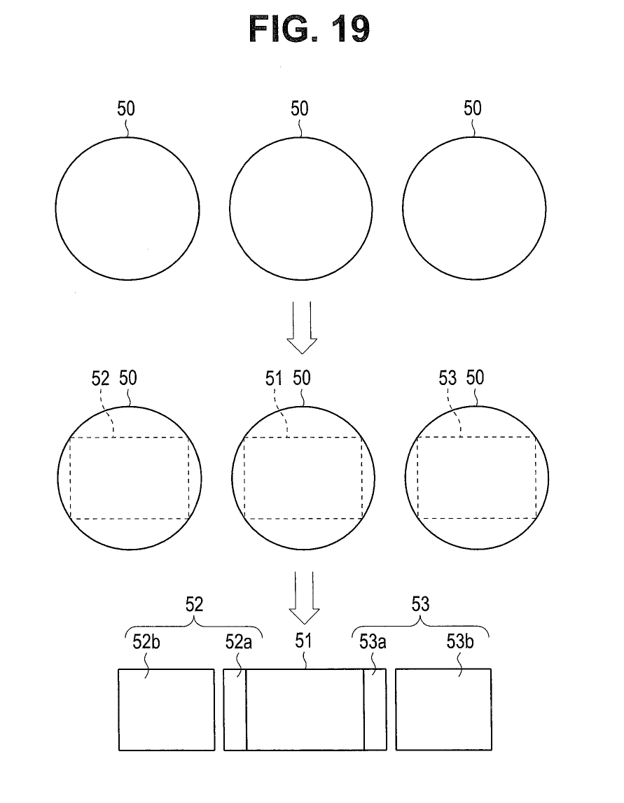

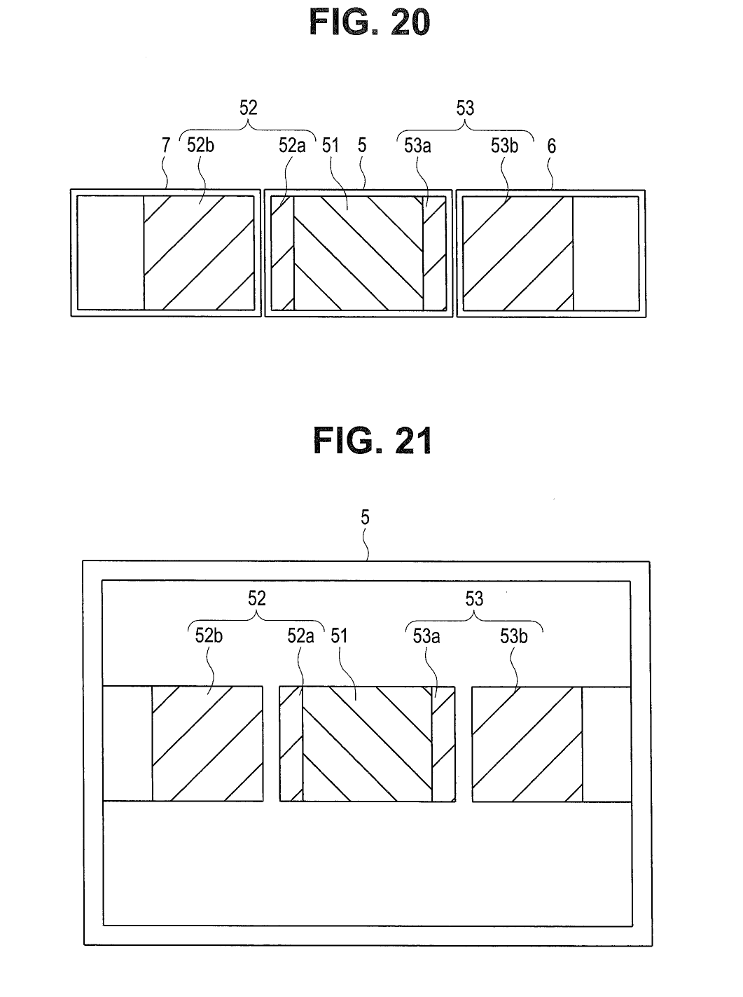

FIG. 18 is a schematic diagram showing a configuration of the processor configured to cause the display apparatuses to display the observation images according to a third modification. FIG. 19 is a diagram describing a procedure of image processing of the third modification. FIG. 20 is a diagram showing a state in which the observation images are displayed on three display apparatuses according to the third modification. FIG. 21 is a diagram showing a state in which the observation images are displayed on one display apparatus according to the third modification.

In the endoscope system 1 of the present modification, the image conversion sections 44, 45, and 46 are not provided on the control section 32 of the processor 3 as shown in FIG. 18. The image dividing and distributing section 36 is further provided in place of the image synthesis section 47, and the monitor 38 is further provided in addition to the three monitors 5, 6, and 7.

In the endoscope system 1 of the present modification configured in this way, the three image cut-out sections 41, 42, and 43 of the control section 32 of the processor 3 cut out the round images of the three captured image regions 50 in a rectangular shape as shown in FIG. 19.

In the control section 32, the image dividing and distributing section 36 divides parts of the lateral image regions 52 and 53 and converts the image regions 52 and 53 to allow displaying the image regions 52 and 53 along with the forward image region 51 (note that synthesis of only the parts is also possible).

In this way, the endoscope system 1 displays the forward image region 51 in the first display region of the monitor 5 among the three monitors 5, 6, and 7 and displays the partial observation images 52a and 53a of the lateral image regions 52 and 53 in the second display region as shown in FIG. 20. The endoscope system 1 displays the remaining lateral observation images 52b and 53b on the monitors 6 and 7.

The endoscope system 1 also records the processed images here in the image recording section 48 provided on the control section 32 of the processor 3.

According to the configuration, the endoscope system 1 can prevent part of the both lateral images from being hidden at the boundaries of the monitor 5 and the monitors 6 and 7.

Note that although the regions are also set to display one display part on the screen of each of the three monitors 5, 6, and 7 in the endoscope system 1 of the present modification, the processor 3 may set a plurality of, for example three, display parts on one screen of one monitor 38 as shown in FIG. 21, and a switching function for operation in another operation mode may be included, in which the forward field of view image and the lateral field of view images are displayed on the plurality of display parts, respectively.

### Fourth Modification

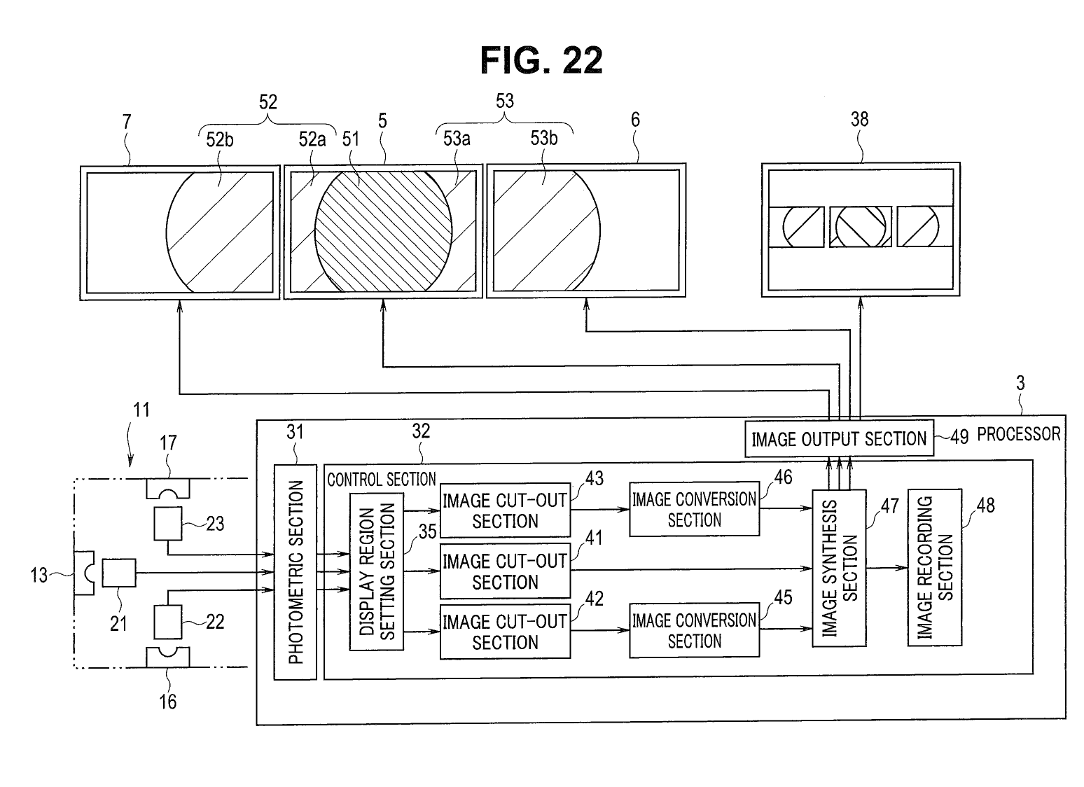

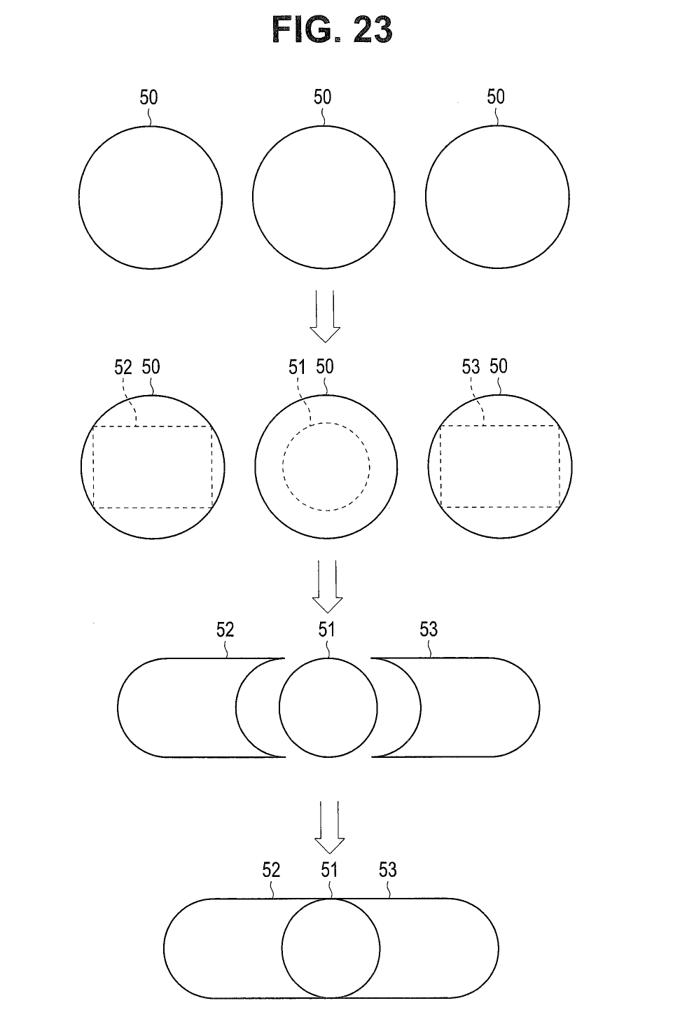

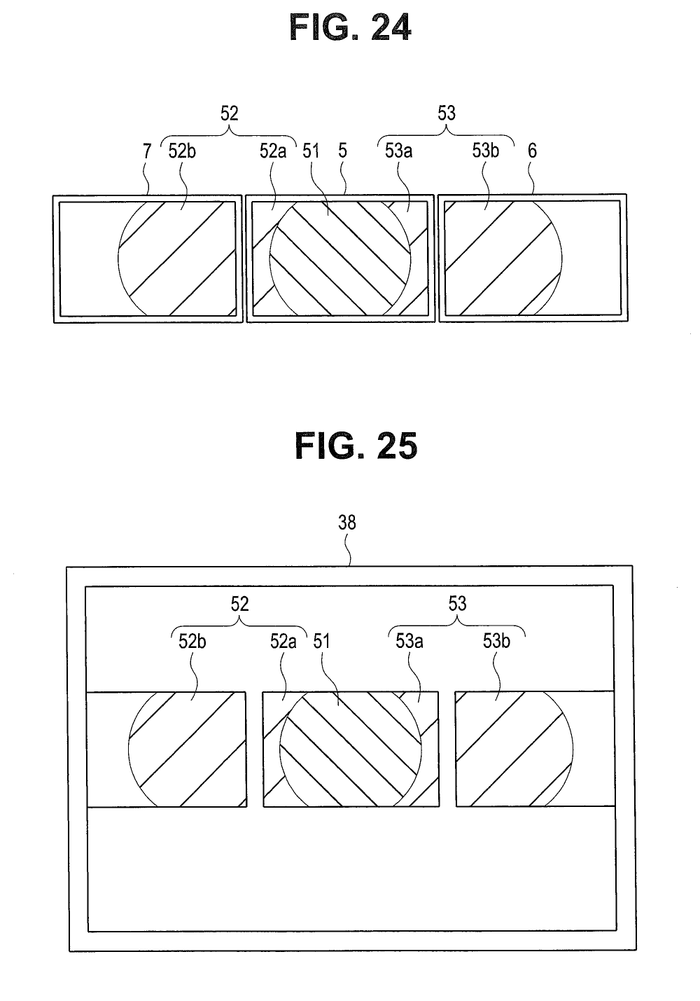

FIG. 22 is a schematic diagram showing a configuration of the processor configured to cause the display apparatuses to display the observation images according to a fourth modification. FIG. 23 is a diagram describing a procedure of image processing of the fourth modification. FIG. 24 is a diagram showing a state in which the observation images are displayed on three display apparatuses according to the fourth modification. FIG. 25 is a diagram showing a state in which the observation images are displayed on one display apparatus according to the fourth modification.

In the endoscope system 1 of the present modification, only two image conversion sections 45 and 46 are provided on the control section 32 of the processor 3, and the monitor 38 is further provided in addition to the three monitors 5, 6, and 7 as shown in FIG. 22.

In the endoscope system 1 of the present modification configured in this way, the control section 32 of the processor 3 causes the image cut-out section 41 to cut out the forward image in a round shape, such as an elliptical shape, a circular shape, and a barrel shape, from the round images of the three captured image regions 50 and causes the image cut-out sections 42 and 43 to cut out the two lateral images in a rectangular shape as shown in FIG. 23.

The control section 32 does not convert the forward image region 51. The image conversion sections 45 and 46 convert the two lateral image regions 52 and 53 into concave shapes, and the image synthesis section 47 synthesizes the three image regions 51, 52, and 53.

In this way, the endoscope system 1 displays the forward image region 51 in the first display region of the monitor 5 and displays the partial observation images 52a and 53a of the lateral image regions 52 and 53 in the second display region as illustrated in FIG. 24.

Note that the remaining observation images 52b and 53b of the lateral image regions 52 and 53 are displayed on the monitors 6 and 7.

The endoscope system 1 also records the processed images in the image recording section 48 provided on the control section 32 of the processor 3.

According to the configuration, the endoscope system 1 substantially correctly displays the dimensions in the horizontal direction of the lateral images displayed in the respective display regions of the three monitors 5, 6, and 7.

Note that although the regions are also set to display one display part on the screen of each of the three monitors 5, 6, and 7 in the endoscope system 1 of the present modification, the processor 3 may set a plurality of, for example three, display parts on one screen of one monitor 38 as shown in FIG. 25, and a switching function for operation in another operation mode may be included, in which the forward field of view image and the lateral field of view images are displayed on the plurality of display parts, respectively.

### Fifth Modification

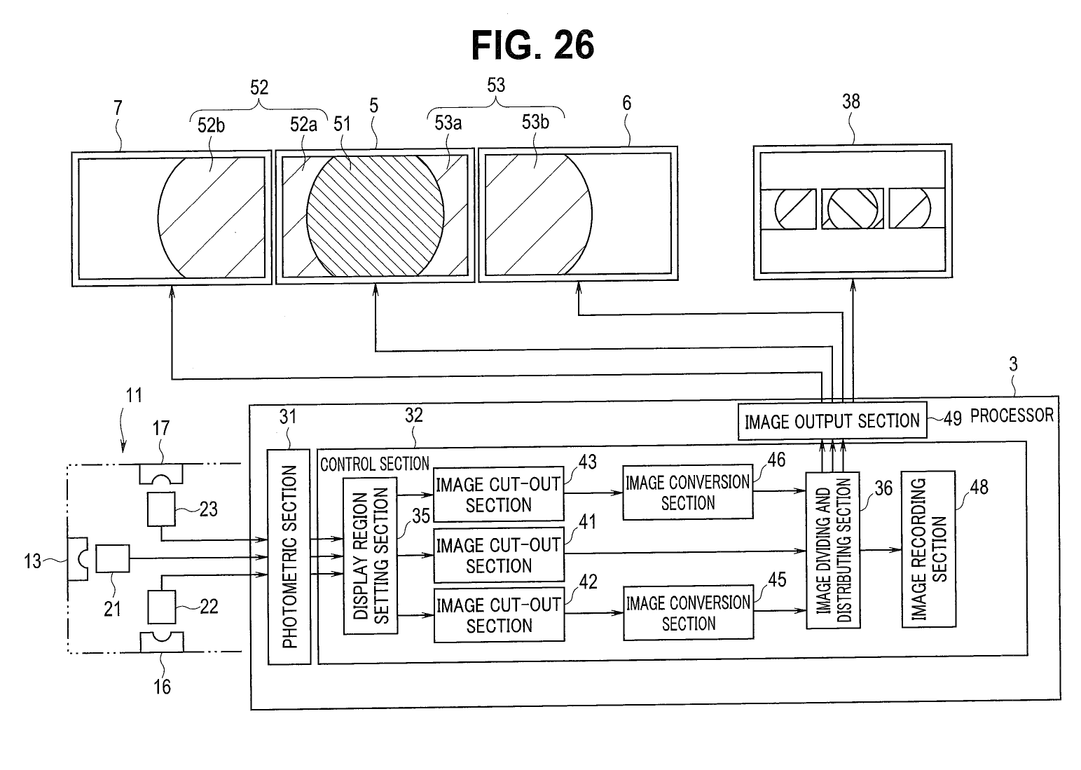

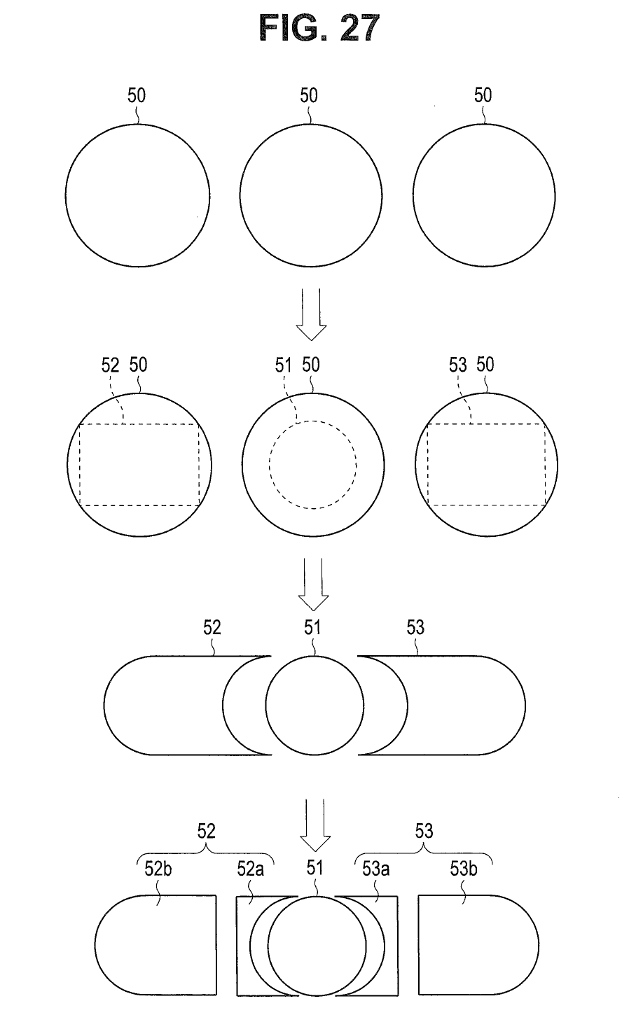

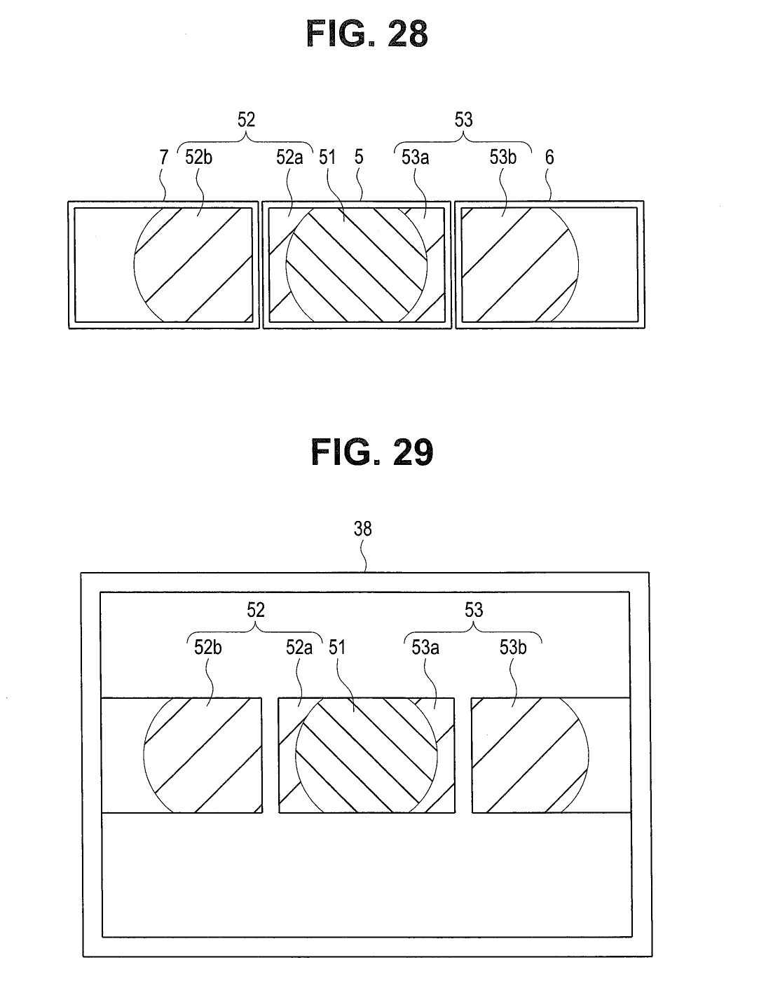

FIG. 26 is a schematic diagram showing a configuration of the processor configured to display the observation images on the display apparatuses according to a fifth modification. FIG. 27 is a diagram describing a procedure of image processing of the fifth modification. FIG. 28 is a diagram showing a state in which the observation images are displayed on three display apparatuses according to the fifth modification. FIG. 29 is a diagram showing a state in which the observation images are displayed on one display apparatus according to the fifth modification.

In the endoscope system 1 of the present modification, only two image conversion sections 45 and 46 are provided on the control section 32 of the processor 3 as shown in FIG. 26. The image dividing and distributing section 36 is further provided in place of the image synthesis section 47, and the monitor 38 is further provided in addition to the three monitors 5, 6, and 7.

In the endoscope system 1 of the present modification configured in this way, the control section 32 of the processor 3 causes the image cut-out section 41 to cut out the forward image in a round shape, such as an elliptical shape, a circular shape, and a barrel shape, from the round images of the three captured image regions 50 and causes the image cut-out sections 42 and 43 to cut out the two lateral images in a rectangular shape as shown in FIG. 27.

In the control section 32, the image conversion sections 45 and 46 convert only the two lateral image regions 52 and 53 into concave shapes.

Subsequently, in the control section 32, the image dividing and distributing section 36 divides parts of the lateral image regions 52 and 53 and converts the image regions 52 and 53 to allow displaying the image regions 52 and 53 along with the forward image region 51 (note that synthesis of only the parts is also possible).

In this way, the endoscope system 1 displays the forward image region 51 in the first display region of the monitor 5 among the three monitors 5, 6, and 7 as shown in FIG. 28. The endoscope system 1 displays the partial observation images 52a and 53a of the lateral image regions 52 and 53 in the second display region and displays the remaining lateral observation images 52b and 53b on the monitors 6 and 7.

The endoscope system 1 also records the processed images in the image recording section 48 provided on the control section 32 of the processor 3.

According to the configuration, the endoscope system 1 can prevent part of both lateral images from being hidden at the boundaries of the monitor 5 and the monitors 6 and 7.

Note that although the regions are also set to display one display part on the screen of each of the three monitors 5, 6, and 7 in the endoscope system 1 of the present modification, the processor 3 may set a plurality of, for example three, display parts on one screen of one monitor 38 as shown in FIG. 29, and a switching function for operation in another operation mode may be included, in which the forward field of view image and the lateral field of view images are displayed on the plurality of display parts, respectively.

### Second Embodiment

Next, an endoscope system of a second embodiment of the present invention will be described based on the drawings. Note that in the following description, the same reference signs are used for the same constituent elements described in the first embodiment, and the detailed description of the constituent elements will not be repeated.

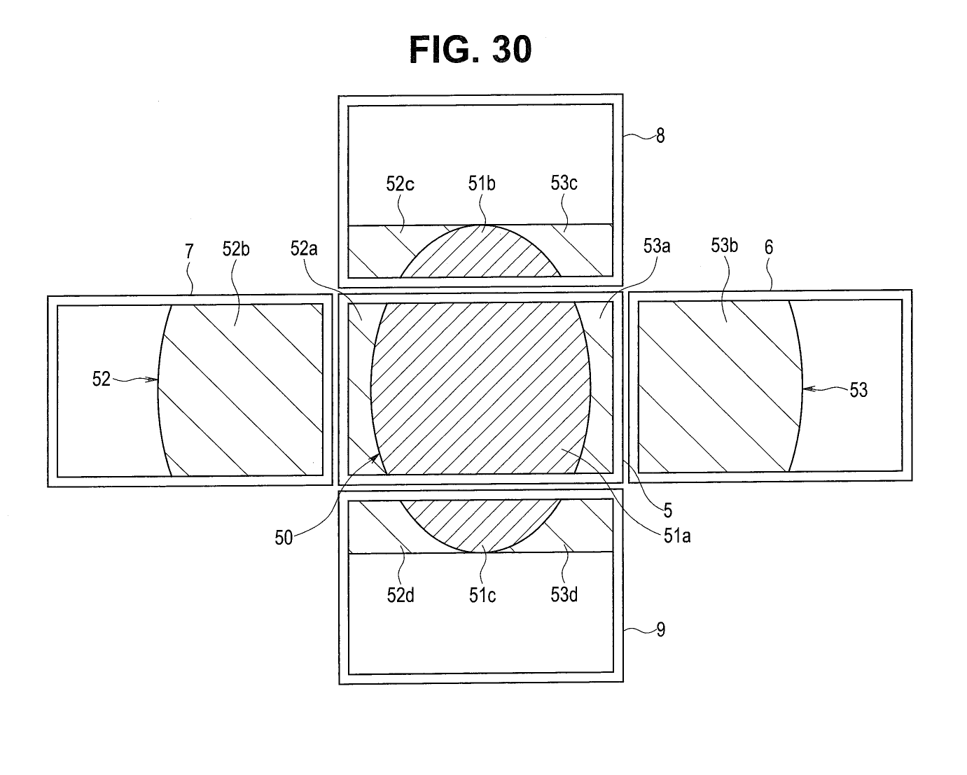

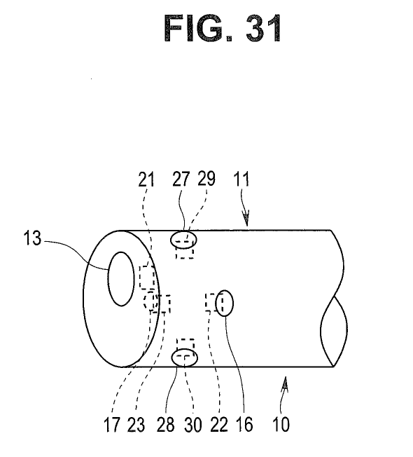

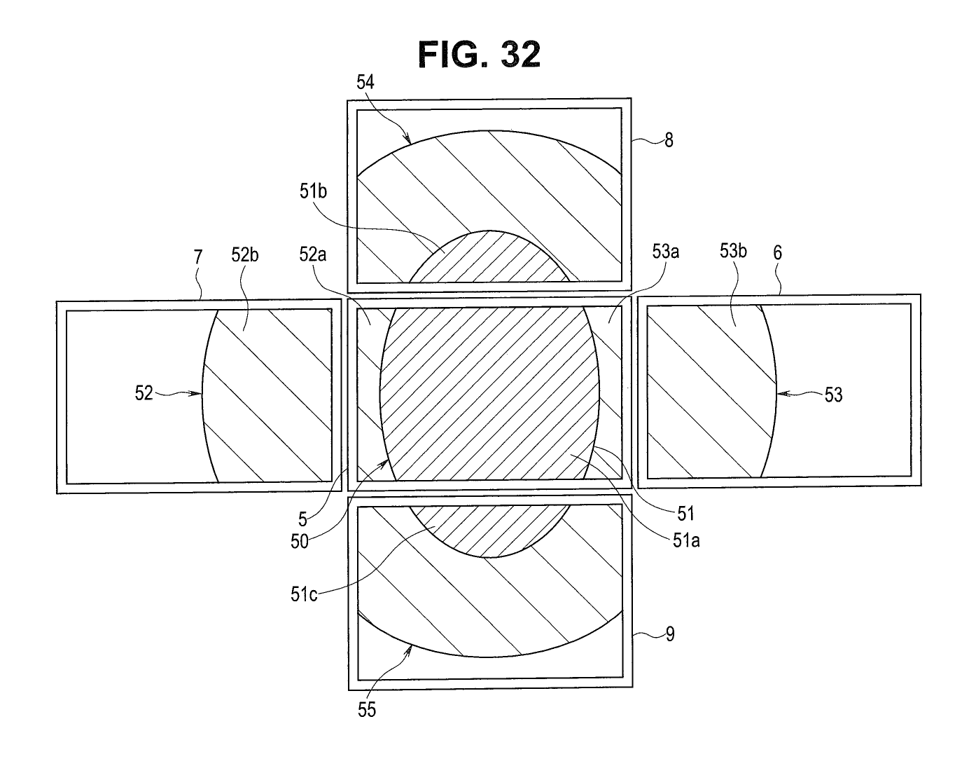

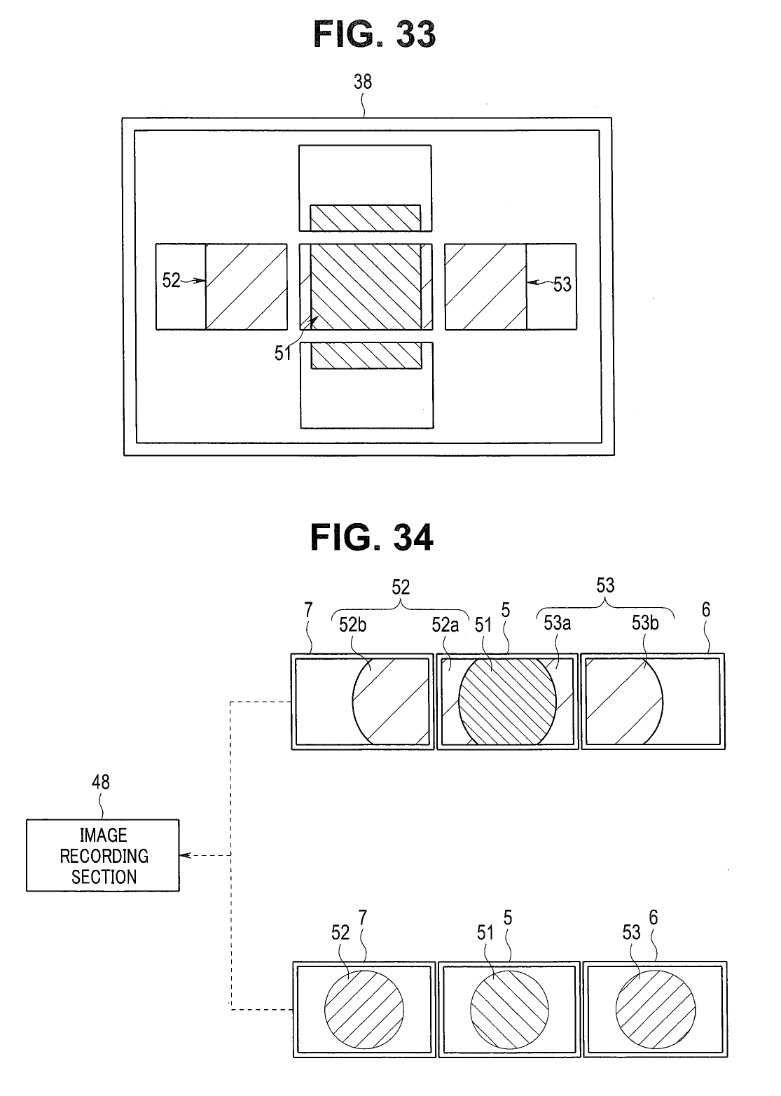

FIG. 30 is a diagram showing five monitors displaying subject images taken by an endoscope. FIG. 31 is a perspective view showing a configuration of the distal end portion of the endoscope according to a modification. FIG. 32 is a diagram showing five monitors displaying the subject images taken by the endoscope according to the modification. FIG. 33 is a diagram showing one monitor displaying the subject image taken by the endoscope according to the modification.

As shown in FIG. 30, the endoscope system 1 of the present embodiment includes monitors 5, 6, 7, 8, and 9 that are five display apparatuses configured to display endoscopic images synthesized by the processor 3 not shown here.

That is, the endoscope system 1 newly includes two monitors 8 and 9 in the up and down direction of the monitor 5 disposed at the center, in addition to the components of the first embodiment.

Up and down parts of the object images acquired by the respective image pickup units 21, 22, and 23 are displayed on the two monitors 8 and 9.

More specifically, an observation image 51a of the image region 51 of the forward field of view and the observation images 52a and 53a of the captured image regions 52 and 53 of the respective lateral fields of view are displayed on the monitor 5 at the center as in the first embodiment.

The remaining partial observation images 52b and 53b excluding the observation images 52a and 53a in the image regions 52 and 53 in the respective lateral field of view directions are displayed on the left and right monitors 6 and 7 as in the first embodiment.

Remaining observation images 51b and 51c in the up and down direction excluding the observation image 51a in the image region 51 of the forward field of view and partial observation images 52c, 52d, 53c, and 53d in the up and down direction excluding the observation images 52a, 52b, 53a, and 53b in the captured image regions 52 and 53 of the respective lateral fields of view are displayed on the up and down monitors 8 and 9.

Here, the up and down direction in the image regions 51, 52, and 53 can be displayed on the up and down monitors 8 and 9, and the three cut-out sections 41, 42, and 43 provided on the control section 32 of the processor 3 do not have to cut out the captured image regions 50 (see FIG. 3) in the up and down direction. Note that the control, such as deforming and synthesizing the observation images, is the same as in the first embodiment.

In the endoscope system 1 configured in this way, the cut-out (masked) parts of the captured image regions 50 as object images acquired by the respective image pickup units 21, 22, and 23, the cut-out parts of which are not used in the display regions of the monitors 5, 6, and 7, can be reduced as much as possible, as compared to the first embodiment. Therefore, the plurality of image pickup units 21, 22, and 23 can effectively show a wide-angle observation image.

The endoscope system 1 is further provided with the monitors 8 and 9 above and below, in addition to the center, left, and right monitors 5, 6, and 7. Therefore, the appearance of the object images displayed on the five monitors 5, 6, 7, 8, and 9 gives a three-dimensional impression, and the visibility can be improved.

Note that if the two monitors 8 and 9 located up and down are always displayed, a large amount of information is in the sight of the user, and the burden of the user increases. Therefore, display and non-display can be switched by hand-side operation of a switch provided on the operation portion or the like of the endoscope 2, foot-side operation of a foot switch, manual operation by a medical assistant, and the like.

Furthermore, the two monitors 8 and 9 may be able to be housed on the rear side or the like of the center monitor 5 or the left and right monitors 6 and 7 when the two monitors 8 and 9 are not necessary.

As a result, the user can make a change for a desired display mode, and the endoscope system 1 can improve the efficiency and the accuracy of endoscopy.

### Modification

Note that although the three observation windows 13, 16, and 17 and the three image pickup units 21, 22, and 23 are provided on the distal end portion 11 of the endoscope 2 in the configuration described above, two observation windows 27 and 28 and two image pickup units 29 and 30 may be further provided on the distal end portion 11 as shown in FIG. 31.

Note that in the distal end portion 11 of the insertion portion 10, the image pickup unit 29 for third lateral field of view is located on the back side of the observation window 27, and the image pickup unit 30 for fourth lateral field of view is located on the back side of the observation window 28.

Each of the image pickup units 29 and 30 is provided on the distal end portion 11 of the insertion portion 10 and is a third image acquisition section configured to acquire a third object image from a direction different from the forward direction that is a first direction and different from the lateral direction that is a second direction in the left and right direction.

In other words, as described above, the first object image is an object image in the first direction including the forward direction of the insertion portion substantially parallel to the longitudinal direction of the insertion portion 10, and the second object image is an object image in the second direction in the left and right direction including the lateral direction of the insertion portion substantially orthogonal to the longitudinal direction of the insertion portion 10. The third object image is an object image in a third direction in the up and down direction substantially orthogonal to the longitudinal direction of the insertion portion 10 and substantially orthogonal to the second direction.

The object images in the forward, up and down, and left and right directions acquired by the five image pickup units 21, 22, 23, 29, and 30 are displayed on the five monitors 5, 6, 7, 8, and 9 as shown in FIG. 32.

More specifically, the observation image 51a of the image region 51 of the forward field of view and the observation images 52a and 53a of the captured image regions 52 and 53 of the first and second lateral fields of view are displayed on the center monitor 5 as described above.

The remaining partial observation images 52b and 53b excluding the observation images 52a and 53a in the image regions 52 and 53 of the first and second lateral field of view directions are displayed on the left and right monitors 6 and 7.

The remaining observation images 51b and 51c in the up and down direction excluding the observation image 51a in the image region 51 of the forward field of view and the image regions 54 and 55 in the third and fourth lateral field of view directions are displayed on the up and down monitors 8 and 9.

In addition to the advantageous effects described above, the endoscope system 1 configured in this way can display an observation image with a wider angle in the up and down direction.

Note that although the regions are set to display one display part on the screen of each of the five monitors 5, 6, 7, 8, and 9 as shown in FIGS. 30 and 32, the processor 3 may set a plurality of, for example five, display parts on one screen of one monitor (38) as shown in FIG. 33, and a switching function for operation in another operation mode may be included, in which the forward field of view image, the lateral field of view images, and the field of view images in the up and down direction are displayed on the plurality of display parts, respectively.

As for the method of cutting out the field of view images in this case, the field of view images may be cut out in a round shape as shown in FIGS. 30 and 32. However, the field of view images may be displayed in, for example, a rectangular state as described in the second and third modifications of the first embodiment.

### Third Embodiment

Next, an endoscope system of a third embodiment of the present invention will be described based on the drawings. Note that in the following description, the same reference signs are used for the same constituent elements described in the first and second embodiments, and the detailed description of the constituent elements will not be repeated. Furthermore, the configuration described below can also be combined with the first and second embodiments.

FIG. 34 is a diagram showing the image recording section and the display apparatus displaying the endoscopic images.

As shown in FIG. 34, the endoscope system 1 here is configured to allow the user to select whether to store the forward image region 51 and the respective lateral image regions 52 and 53 in a filing format of one image or to store the forward image region 51 and the respective lateral image regions 52 and 53 in a filing format of a multiple-recording mode of separately storing the regions and synchronizing the photographing time, during filing in the image recording section 48 provided on the control section 32 of the processor 3.

Note that the selection can be switched by, for example, a switch provided on the operation portion of the endoscope 2 or a switch provided on the processor 3 (the switches are not shown).

According to the configuration, the endoscope system 1 can set a recording mode according to the preference of the user to record the observation images in the filing format corresponding to the recording mode. Therefore, the endoscope system 1 can efficiently search or display the observation images in the respective image regions 51, 52, and 53. As a result, the user can easily review the observation images or create a report.

### Fourth Embodiment

In each of the embodiments and each of the modifications, the mechanism for realizing the function of illuminating and observing the lateral direction is built in the insertion portion 10 along with the mechanism for realizing the function of illuminating and observing the forward direction. However, the mechanism may be a separate body that can be attached to and detached from the insertion portion 10.

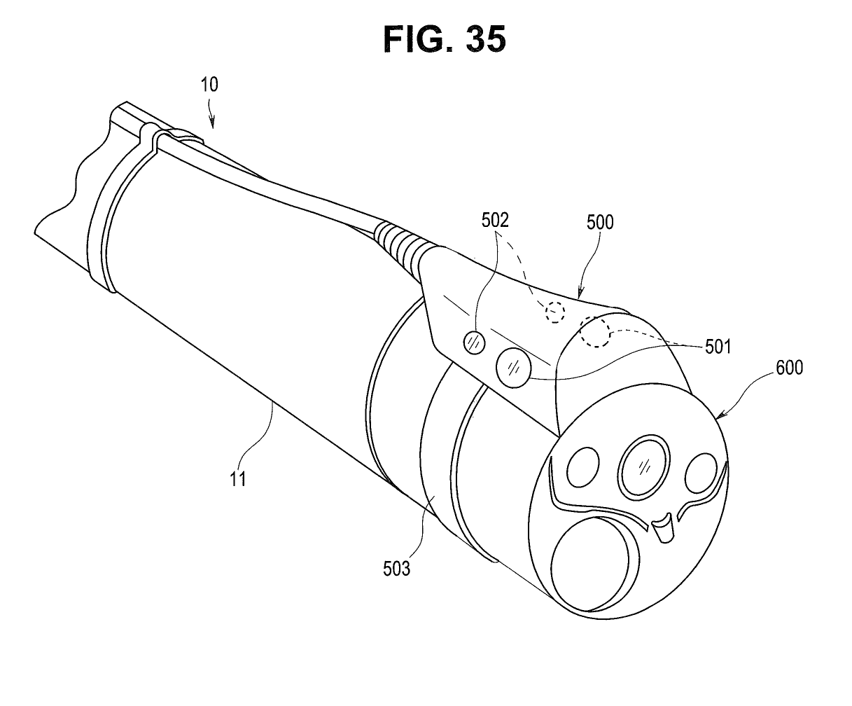

Note that FIG. 35 is a perspective view of the distal end portion 11 of the insertion portion 10 provided with a unit for lateral observation according to a fourth embodiment.

The distal end portion 11 of the insertion portion 10 includes a lateral field of view unit 500. The lateral field of view unit 500 can be attached to and detached from a forward field of view unit 600 through a clip portion 503.

The lateral field of view unit 500 includes two observation windows 501 for acquiring images in the left and right direction and two illumination windows 502 for illuminating the left and right direction.

The processor 3 and the like can turn on and turn off each of the illumination windows 502 of the lateral field of view unit 500 according to a frame rate of the forward field of view to acquire and display the observation images as illustrated in the embodiments.

As described, the respective embodiments and modifications can provide an endoscope system that allows quick observation when the field of view direction of the endoscope with a wide-angle field of view is changed.

The invention described in the embodiments is not limited to the embodiments and the modifications of the invention, and various modifications can also be carried out in an execution phase without departing from the scope of the invention. Furthermore, the embodiments include inventions of various phases, and various inventions can be extracted based on appropriate combinations of a plurality of disclosed constituent conditions.

For example, when the problems can be solved and the advantageous effects can be obtained even if some of the constituent conditions illustrated in the embodiments are deleted, the configuration after the deletion of the constituent conditions can be extracted as an invention.

What is claimed is:

1. An endoscope system comprising:
a first image acquisition section configured to acquire a first image from a first region of an object;
a second image acquisition section configured to acquire a second image from a second region of the object including a region adjacent to the first region;
an image synthesis section configured to divide the second image into an adjacent image adjacent to the first image and an image of another region, generate a synthetic image by synthesizing the first image and the adjacent image so as to dispose the adjacent image adjacent to the first image, and generate the image of the other region as an image different from the synthetic image; and
an image output section configured to output the images from the image synthesis section, wherein
the image output section is provided with:
a multiple-display mode of individually converting the synthetic image and the image of the other region into display signals, outputting, to a first display apparatus, the display signal obtained by converting the synthetic image, and outputting, to a second display apparatus, the display signal obtained by converting the image of the other region; and
a single-display mode of generating, based on the synthetic image and the image of the other region, a display signal indicating a video including the synthetic image and the image of the other region disposed separately from each other and outputting the display signal to a display apparatus, and
the image output section switches one of the multiple-display mode and the single-display mode to output the display signal.

2. The endoscope system according to claim 1, further comprising:
a third image acquisition section configured to acquire a third image from a third region of the object adjacent to the first region from a direction different from the second region, wherein
the image synthesis section generates the adjacent image from the second image and the third image and synthesizes the adjacent image with the first image to generate the synthetic image.

3. The endoscope system according to claim 1, wherein
the first image acquisition section comprises a first image pickup section configured to photoelectrically convert the first image, and
the second image acquisition section comprises a second image pickup section different from the first image pickup section, the second image acquisition section being configured to photoelectrically convert the second image.

4. The endoscope system according to claim 1, wherein
the first image acquisition section and the second image acquisition section are provided on an insertion portion,
the first region is a region including a forward direction of the insertion portion in a longitudinal direction of the insertion portion, the first image is an image of the first region acquired by the first image acquisition section,
the second region is a region including a lateral direction of the insertion portion orthogonal to the longitudinal direction of the insertion portion, and the second image is an image of the second region acquired by the second image acquisition section.

5. The endoscope system according to claim 1, further comprising:
an image conversion section configured to deform at least one of the first image and the second image into a predetermined shape.

6. The endoscope system according to claim 5, wherein
the image conversion section converts a side end portion of the first image into a convex curved shape and converts the second image into a distorted shape that is a concave curved shape corresponding to the convex curved shape of the first image and adjacent to the first image.

7. The endoscope system according to claim 1, further comprising:
an image recording section configured to record the first image and the second image.

8. The endoscope system according to claim 7, further comprising:
a switch configured to selectively switch
a first mode of synthesizing the first image and second image into one image and storing the image in the image recording section in a one-filing format,
a second mode of storing the first image and the second image in the image recording section in a multiple-filing format to separately store the first image and the second image, and
a third mode of simultaneously storing, in the image recording section, the first image and the second image synthesized in the one-filing format and the first image and the second image separately stored in the multiple-filing format.
